(19) United States
(12) Patent Application Publication
(10) Pub. No.: US 2004/0097583 A1
(43) Pub. Date: May 20, 2004

(54) Use of inhibitors of the sodium/hydrogen exchanger for the treatment of thrombotic and inflammatory disorders

(76) Inventors: Hans-Jochen Lang, Hofheim (DE); Stefan Werner Schneider, Munster (DE); Hans Oberleithner, Munster (DE); Andre Niemeyer, Munster (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/360,943

(22) Filed: Feb. 7, 2003

Related U.S. Application Data
(60) Provisional application No. 60/366,743, filed on Mar. 22, 2002.

(30) Foreign Application Priority Data
Feb. 14, 2002 (DE) 10206358.3

(51) Int. Cl. A61K 31/255; A61K 31/165

(52) U.S. Cl. 514/518; 514/618

(57) ABSTRACT

Inhibitors of the cellular sodium/hydrogen exchangers show an inhibiting effect on the secretion of von-Willebrand factor and/or increased expression of P-selectin. These inhibitors can therefore be employed for the treatment of thrombotic and inflammatory disorders.

### CROSS-REFERENCE TO RELATED APPLICATION

This application claims priority from U.S. Provisional Patent Application No. 60/366,743, filed Mar. 22, 2002 as well as from German Patent Application No. 10206358.3, filed Feb. 14, 2002.

### SUMMARY OF THE INVENTION

The invention relates to the use of inhibitors of the cellular sodium/hydrogen exchanger in human and veterinary medicine for the prevention and treatment of acute or chronic diseases caused by elevated levels of von Willebrand factor in the blood. The inhibitors can therefore be employed for the treatment of thrombotic and inflammatory disorders.

### BACKGROUND OF THE INVENTION

Inhibitors of the sodium/hydrogen exhanger (NHE) have in recent years been characterized in numerous preclinical studies as substances which are suitable in a superior manner in cases of cardiac hypoperfusion for protecting the cardiac tissue, which is endangered by the acute onset of the ischemic event, from death. Protection of cardiac tissue by NHE inhibitors encompasses all degrees of harm caused by the hypoperfusion, starting from cardiac arrhythmias via hypercontraction of the myocardium and temporary loss of function up to death of cardiac tissue and the permanent damage associated therewith.

The mechanism of action of NHE inhibitors which is important in the acute ischemic event comprises their reduction of the enhanced influx of sodium ions which arises in acutely hypoperfused tissue due to activation of the NHE as a consequence of intracellular acidification. This delays the situation of tissue sodium overload. Since there is coupling of sodium and calcium ion transport in cardiac tissue, this prevents the life-threatening calcium overload of heart cells.

It is also known that NHE inhibitors provide protection to the central nervous system (CNS), such agents protecting the CNS, in a similar way to the heart, against acute ischemic states. These states are caused by acute hypoperfusion and thus by a deficient supply of nutrients, oxygen or minerals. Such ischemic harm to the CNS is particularly pronounced in cases of central infarctions such as stroke.

Thus, as expected, no protective effects of NEE inhibitors against these acute events were observable where blood flow was normal and healthy, because there was no acute onset of ischemic harm to cardiac tissue or CNS tissue.

Numerous classes of substances which intervene in the interplay of coagulation factors and thus cause cessation of the coagulation cascade are described in the prior art. Likewise, numerous action principles which do not suppress thrombus formation, but cause the dissolution (lysis) of thrombi which have already formed, have been developed. Some of these action principles, which intervene at a wide variety of junction points in said cascade, have been introduced into therapy to prevent thrombogenesis. These include derivatives of the vitamin K group (phylloquinones), factor VIII and factor IX products, platelet aggregation inhibitors such as acetylsalicylic acid, dipyridamole and ticlopidine, and anticoagulants such as heparins or heparinoids.

The blood coagulation cascade can be divided mechanistically into two pathways as depicted in the following diagram, namely, into an intrinsic and an extrinsic route, the two of which finally meet in the activation of factor X and the resulting generation of thrombin and subsequently of fibrin:

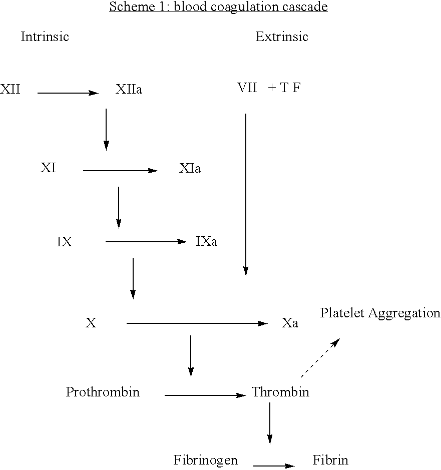

It is important in the therapeutic use of such blood coagulation inhibitors that the inhibition of coagulation achieved is not too strong or complete, which would inhibit the formation of microthrombi and microcoagulations, which are vital and which must take place at the microtraumata, which are continually happening. Only imprecise adjustment of the degree of inhibition of coagulation is possible as a result of differences in the response of the particular individual at a particular time, and the degree must be carefully monitored where possible. If these many small coagulation processes, which are permanently taking place, are inhibited, there is a high risk of extensive hemorrhage (hemophilia).

The disadvantage of the known therapeutic agents available on the market, which intervene as inhibitors in the coagulation event, is, therefore, the high risk of bleeding complications. The risk of life-threatening hemorrhage exists especially during high-dose thrombolysis therapy, e.g., during therapy of acute myocardial infarction or pulmonary embolism. There is thus an urgent need for therapeutic agents which do not entail a risk of increased tendency to bleeding despite overdose.

Many of the known anticoagulant substances act by exerting an effect on the blood platelets, the thrombocytes, and inhibiting their function or inhibiting their activation. The endothelium also evidently plays a central part in the coagulation event. Thus, for example, the von Willebrand factor (vWF), which is necessary for coagulation, is produced for the most part in endothelial cells and is secreted by them permanently (constitutively) into the circulating blood in order to ensure the necessary coagulation processes in the blood. A considerable part of the produced vWF is stored in cytoplasmic granules, called Weibel-Palade bodies, and released as required through stimulation of endothelial cells. If endothelial cells are unable to produce vWF and deliver it to the blood, the result is the well known genetic, vWF-dependent disease, von Willebrand-Jürgens syndrome, which is characterized by hemorrhages which can scarcely be stopped.

It is only in recent years that disorders caused by elevated concentrations of vWF in the blood, which would induce, for example, an increased tendency to blood coagulation and inflammatory processes, have become known. Thus, Kamphuisen et al. demonstrate on the basis of a large number of studies, in their publication “Elevated factor VIII levels and the risk of thrombosis” (Arterioscler. Thromb. Vasc. Biol. 21(5):731-738 (2001)), that there is a significant association between elevated vWF levels in the blood and an increased rate of thrombotic disorders. Factor VIII forms a complex with vWF as a necessary precondition for blood coagulation. It has been possible to establish that high levels of von Willebrand factor (vWF) and of vWF-bound factor VIII in the blood represent a clear thrombosis risk factor. However, antithrombotic agents which antagonize the stabilizing binding of vWF to factor VIII may also be disadvantageous because, in the event of overdosage, substantial inhibition of blood coagulation and dangerous tendencies to bleeding must be expected.

### DETAILED DESCRIPTION OF THE INVENTION

In an effort to find effective compounds for the treatment of acute or chronic diseases caused by elevated levels of von Willebrand factor in the blood, it has now been found that the compounds described below, when employed according to the invention, inhibit the release of von Willebrand factor from endothelial cells. The compounds of the invention inhibit the massive pH-dependent release of vWF, which accumulates during ischemia.

Whereas the secretion takes place normally and constitutively at the normal pH of blood, which is known to be about 7.4, and part of the vWF is stored in Weibel-Palade bodies, it has now been found that there is a delay and reduction in the release of vWF as the pH falls. Exocytosis of the Weibel-Palade bodies in which the vWF is packaged is increasingly inhibited as the pH declines. Thus, under acidotic conditions, there is a significant increase in Weibel-Palade bodies and, thus, extensive accumulation of vWF in the endothelial cell, and a reduced constitutive and stimulated vWF secretion. This can be visualized by staining procedures and demonstrated by quantitative measurements of vWF in the supernatant. Such acidotic states with significant pH reductions below 7 occur, for example, in cases of tissue ischemia. At the instant of realkalinization and endothelial cell stimulation, which corresponds to the reperfusion state, within seconds, exocytosis takes place, with consequent emptying of the Weibel-Palade bodies (WPB), thus leading to massive release of the prothrombotic risk factor.

Besides vWF, the Weibel-Palade bodies also store the transmembrane protein P-selectin (Wagner, D. D. 1993, Thromb. Haemost., 70:105-110). P-Selectin is located in the vesicle membrane and, after vesicle fusion (exocytosis), is incorporated into the plasma membrane of the endothelial cell. This means that every Weibel-Palade body exocytosis leads not only to increased vWF release but also to increased P-selectin expression in the endothelial cell membrane. The following examples show vWF secretion (quantitive measurement by ELISA) during acidosis and during subsequent reperfusion. In parallel, these quantitative measurements are confirmed by immunofluorescence data on the Weibel-Palade bodies. The measured vWF is, thus, not only a marker of increased (increase in vWF secretion) or reduced (decrease in vWF secretion) tendency to thrombosis (via increase in platelet aggregation), but also a direct marker of increased or reduced P-selectin expression in the endothelial cell membrane. P-Selectin serves as an anchor for leukocytes and, therefore, the initial inflammatory reaction (Vestweber, D., Blanks, J. E. 1999, Physiol. Rev., 79:181-213; Issekutz, A. C., Issekutz, T. B. 2002, J. Immunol., 168:1934-1939). The pathophysiological significance is wide-ranging and confirmed for ischemia/reperfusion disorders, thromboses and arteriosclerosis (Massberg, S., et al., 1998, Blood, 92:507-515; Kita, T., et al., 2001, Ann. N. Y. Acad. Sci., 947:199-205). Besides the significance of P-selectin as a marker of inflammation and initiator of inflammation, it plays an essential part in the process of cancer dissemination (Varki, A., Varki, N. M. 2001, Braz. J. Med. Biol. Res. 34:711-717) as well as during various inflammations of joints (arthritis) (Veihelmann, A. et al, 1999, Microcirculation, 6: 281-290; McInnes, I. B., et al., 2001, J. Immunol., 167:4075-4082). Thus the mode of action of the substances described herein may also find use as therapy for all the abovementioned P-selectin-associated disorders.

The invention, therefore, relates to the use of inhibitors of the sodium/hydrogen exchange for the prophylaxis and therapy of acute and chronic diseases caused by elevated levels of von Willebrand factor in blood.

The invention further relates to the use of at least one of the following compounds

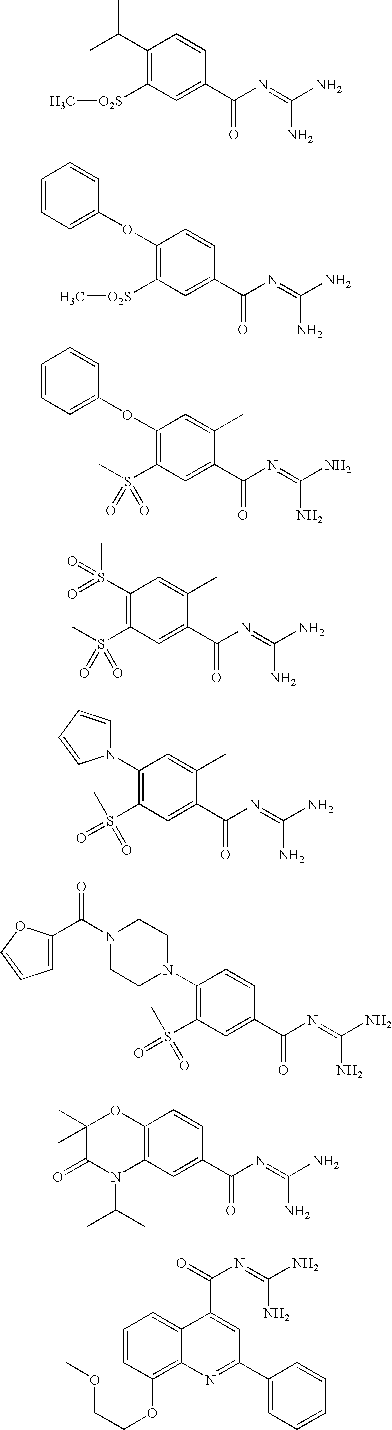

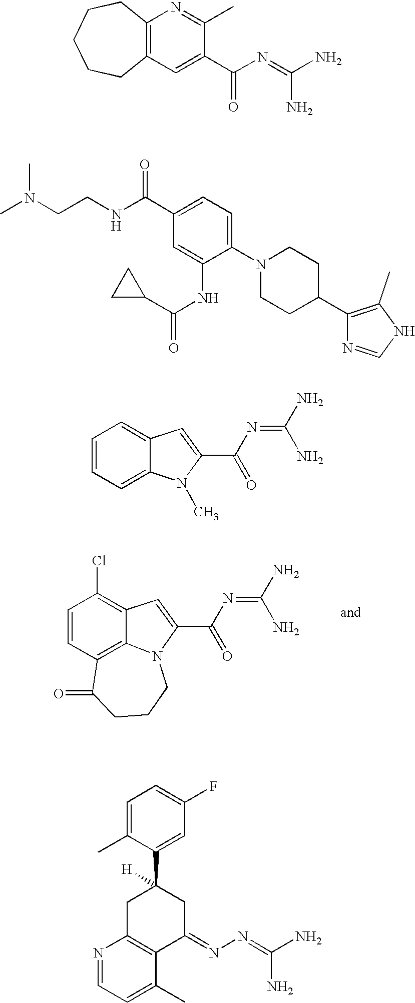

and/or stereoisomeric forms of the abovementioned compounds and/or mixtures of these forms in any ratio, and/or of the physiologically tolerated salts of the abovementioned compounds for the prophylaxis and therapy of acute or chronic diseases caused by elevated levels of von Willebrand factor in the blood and/or increased expression of P-selectin.

The invention further relates to the use of cariporide

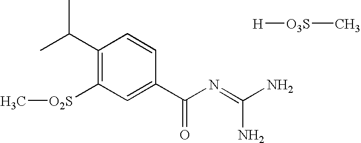

for the prophylaxis and therapy of acute or chronic diseases which are caused by elevated levels of von Willebrand factor in the blood and/or increased expression of P-selectin.

The abovementioned compounds are known and can be prepared as described, for example, in EP 0 416 499, EP 0 556 673, EP 0 589 336, EP 0 622 356, EP 0 699 666, EP 0 708 088, EP 0 719 766, EP 0 726 254, EP 0 787 728, EP 0 972 767, DE 19529612, DE 19601303, WO 99 00379 or T. Kawamoto et al., Potent and selective Inhibition of the human Na+/H+ exchanger isoform NHE1 by a novel aminoguanidine derivative T-162559, Eur. J. Pharmacol. 420 (2001), 1-8.

Where the abovementioned compounds have diastereoisomeric or enantiomeric forms and result as mixtures thereof in the chosen synthesis, separation into the pure stereoisomers takes place either by chromatography on an optionally chiral support material or, if the abovementioned racemic compounds are able to form salts, by fractional crystallization of the diastereomeric salts formed with an optically active base or acid as an aid. Examples of suitable chiral stationary phases for separation of enantiomers by thin-layer or column chromatography are modified silica gel supports (so-called Pirkle phases) and high molecular weight carbohydrates such as triacetylcellulose. Gas chromatographic methods on chiral stationary phases can also be used for analytical purposes after appropriate derivatization known to the skilled art worker. To separate enantiomers of the racemic carboxylic acids, diastereomeric salts differing in solubility are formed (using an optically active, usually commercially available, base such as (−)-nicotine, (+)- and (−)-phenylethylamine, quinine bases, L-lysine or L- and D-arginine), the less soluble component is isolated as a solid, the more soluble diastereomer is deposited from the mother liquor, and the pure enantiomers are obtained from the diastereomeric salts obtained in this way. It is possible, in the same way, in principle, to convert the racemic compounds of the formula I containing a basic group, such as an amino group, with optically active acids, such as, (+)-camphor-10-sulfonic acid, D- and L-tartaric acid, D- and L- lactic acid and (+) and (−)-mandelic acid into the pure enantiomers. Chiral compounds containing alcohol or amine functions can also be converted with appropriately activated or, where appropriate, N-protected enantiopure amino acids into the corresponding esters or amides, or, conversely, chiral carboxylic acids can be converted with carboxyl-protected enantiopure amino acids into the amides or with enantiopure hydroxy carboxylic acids, such as lactic acid, into the corresponding chiral esters. The chirality of the amino acid or alcohol residue produced in enantiopure form can then be utilized for separating the isomers by carrying out a separation of the diastereomers, which are now present, by crystallization or chromatography on suitable stationary phases and then eliminating the included chiral moiety by suitable methods.

Acidic or basic products of the abovementioned compounds can exist in the form of their salts or in free form. Preference is given to pharmacologically suitable salts, e.g. alkali metal or alkaline earth metal salts, or hydrochlorides, hydrobromides, sulfates, hemisulfates, all possible phosphates, and salts of amino acids, natural bases or carboxylic acids.

Physiologically tolerated salts are prepared from the abovementioned compounds that are able to form salts, including the stereoisomeric forms thereof, in a manner known per se. The salts are formed, for example, by interaction of carboxylic acids and hydroxamic acids with basic reagents such as hydroxides, carbonates, bicarbonates, alcoholates and ammonia or organic bases, for example, trimethyl- or triethylamine, ethanolamine or triethanolamine or else basic amino acids, for example lysine, ornithine or arginine, stable alkali metal, alkaline earth metal or optionally substituted ammonium salts. Where the abovementioned compounds have basic groups, stable acid addition salts can also be prepared with strong acids. Suitable for this purpose are both inorganic and organic acids, such as hydrochloric, hydrobromic, sulfuric, phosphoric, methanesulfonic, benzenesulfonic, p-toluenesulfonic, 4-bromobenzenesulfonic, cyclohexylsulfamic, trifluoromethylsulfonic, acetic, oxalic, tartaric, succinic and trifluoroacetic acid. Methanesulfonic acid salts of the abovementioned compounds are particularly preferred.

Owing to their pharmacological properties, the abovementioned compounds are suitable for the prophylaxis and therapy of acute or chronic diseases which are caused by elevated levels of von Willebrand factor in the blood and/or increased expression of P-selectin. These include thrombotic disorders provoked by ischemic states with subsequent reperfusion, such as thromboses in acute myocardial, mesenteric or else cerebral infarction; thrombotic disorders occurring during or after surgical operations; pulmonary embolisms; deep vein thromboses such as occur at an increased rate after prolonged restriction of blood flow, especially in the lower extremities, for example after prolonged lying or sitting, and inflammatory disorders such as occur during ischemia and subsequent reperfusion, or during vasculitis (e.g. associated with autoimmune disease or connective tissue disease). These also include disorders which are caused by increased expression of P-selectin, such as incipient inflammatory reactions, but also prophylaxis and treatment of arteriosclerosis; and prophylaxis and treatment of cancer; also inflammation of joints and arthritic disorders such as rheumatoid arthritis.

Administration of the medicaments of the invention can take place by oral, inhalational, rectal or transdermal administration or by subcutaneous, intraarticular, intraperitoneal or intravenous injection. Oral administration is preferred.

The abovementioned compounds are mixed with the additives, such as carriers, stabilizers or inert diluents, and converted by conventional methods into suitable dosage forms such as tablets, coated tablets, two-piece capsules, aqueous, alcoholic or oily suspensions or aqueous or oily solutions. Examples of inert carriers which can be used are gum arabic, magnesia, magnesium carbonate, potassium phosphate, lactose, glucose or starch, especially corn starch. Preparation can moreover take place both as dry and as wet granules. Examples of suitable oily carriers or solvents are vegetable or animal oils, such as sunflower oil or fish liver oil.

For subcutaneous, intraperitoneal or intravenous administration, the active compounds are converted into solution, suspension or emulsion, if desired, with the substances suitable for this purpose, such as solubilizers, emulsifiers or other excipients. Examples of suitable solvents are physiological saline or alcohols, e.g. ethanol, propanol, glycerol, as well as sugar solutions, such as glucose or mannitol solutions, or else a mixture of the various solvents mentioned.

Also used are conventional aids such as carriers, disintegrants, binders, coating agents, swelling agents, glidants or lubricants, flavorings, sweeteners and solubilizers. Excipients which are frequently used and which may be mentioned are magnesium carbonate, titanium dioxide, lactose, mannitol and other sugars, talc, milk protein, gelatin, starch, cellulose and derivatives thereof, animal and vegetable oils such as fish liver oil, sunflower, peanut or sesame oil, polyethylene glycol and solvents such as, for example, sterile water and monohydric and polyhydric alcohols such as glycerol.

The abovementioned compounds are preferably produced and administered as pharmaceutical products in dosage units, where one unit contains as active ingredient a defined dose of the compound of formula I. For this purpose, they can be administered orally in doses of from 0.01 mg/kg/day to 25.0 mg/kg/day, preferably 0.01 mg/kg/day to 5.0 mg/kg/day or parenterally in doses of from 0.001 mg/kg/day to 5 mg/kg/day, preferably 0.001 mg/kg/day to 2.5 mg/kg/day. The dosage may also be increased in severe cases. However, lower doses also suffice in many cases. These data relate to an adult weighing about 75 kg.

The abovementioned compounds can be employed alone or in combination with anticoagulant, platelet aggregation-inhibiting or fibrinolytic agents. Coadministration can take place, for example, with factor Xa inhibitors, standard heparin, low molecular weight heparins such as enoxaparin, dalteparin, certroparin, parnaparin or tinzaparin, direct thrombin inhibitors such as hirudin, aspirin, fibrinogen receptor antagonists, streptokinase, urokinase and/or tissue plasminogen activator (tPA).

It is known that the inhibitors of the sodium/hydrogen exchanger affect platelet aggregation and have an adhesion-inhibiting effect (see Rosskopf, Dieter, J. Thromb. Thrombolysis (1999), 8(1), 15-23; or Nieuwland, Rienk; Akkerman, Jan-Willem Nicolaas. Adv. Mol. Cell Biol. (1997), 18(Platelet), 353-366.

In contrast to the previously described effects on the aggregation of blood platelets, the abovementioned compounds also show inhibition of excessive release of von Willebrand factor. This novel antithrombotic action principle differs from the previously disclosed antithrombotic action principles in a crucial and advantageous manner in that

a) it acts only in ischemic tissue in the subsequent reperfusion phase, whereas other cells not affected by the ischemia (preischemic) remain completely unaffected, and

b) there is no need to worry about any of the dangerous hemorrhagic complications during the lysis therapy.

The invention is explained in more detail by means of the examples set forth below.

The following examples demonstrated the effects of an extracellular acidosis (pHₑₓ=6.4) and the effects of the abovementioned compounds of the invention on the intracellular pH (pHᵢ) and the release of von-Willebrand factor (vWF). All the examples were carried out with human umbilical vein endothelial cells (HUVEC). These comprise primary cell cultures isolated from the umbilical vein.

For the following examples, the cells were cultivated either on gelatinized glass plates (measurement of the intracellular proton concentration) or on cell culture plates (12-well culture plates, Falcon, N.J., USA; measurement of vWF release) after the first passage.

### EXAMPLE 1

### Measurement of the Intracellular pH

To measure the intracellular proton concentration (pHᵢ), the HUVECs were loaded with the pH-sensitive fluorescent dye, BCECF-AM (2′,7′-bis(carboxyethyl)-5(6)-carboxyfluorescein). A Deltascan spectrofluorometer (PTI, Hamburg) was employed for the subsequent fluorescence measurement. This measuring system consists essentially of a UV light source, a monochromator, a photon detector and the Felix and Oscar software packages (PTI, Hamburg) for controlling the system via a computer. After alternate excitation with the wavelengths 439.5 nm (pH-independent) and 490 nm (pH-sensitive), the ratio of the measured emissions of the BCECF (ratio) was reported and the pH was found after a calibration. The measuring cell is designed so that the parameters of temperature and carbon dioxide partial pressure in the system are controlled during continuous perfusion. For the reperfusion simulation, the experimental conditions were set at 37° C. and a carbon dioxide partial pressure of 5% or 10% by gassing the system and perfusate.

In the experiment, there was initially preincubation with sodium bicarbonate buffer pHₑₓ 6.4 for 60 minutes in order to simulate respiratory metabolic acidosis. The initiation perfusion was then changed to sodium bicarbonate buffer of pH 7.4 with 10 μM histamine as reperfusion simulation. These control experiments were compared with an experiment in which the NHE inhibitor cariporide was added in a concentration of 10 μM to the reperfusion buffer.

The results of several experiments have been summarized in Tables 1 and 2.

Table 1: Intracellular pH during extracellular acidosis (pHᵢ (acidosis)) of at least 15 minutes and under control conditions (Co).

TABLE 1

| | |
|---|---|
| pHᵢ (Acidosis) | 6.53 ± 0.02 (mean ± SEM) |
| pHᵢ (Co) | 7.23 ± 0.02 (mean ± SEM) |

SEM is the standard deviation from the mean

Extracellular acidosis leads to intracellular acidification, which persists during the acidosis. The intracellular acidotic pH is virtually identical to the extracellular pH (applied extracellular acidosis pHₑₓ=6.4).

Table 2: Reperfusion with experimental buffer containing cariporide (HOE) and control buffer (Co). The initial rates of increase in the pHᵢ values were found after 60 minutes of acidosis from the measurements during the first 30 seconds after reperfusion.

TABLE 2

Rate of pH increase [Δ pH / min]

| | Individual experiments | Mean ± SEM |
|---|---|---|
| Co | 0.97 | 0.97 ± 0.04 |
| | 1.04 | |
| | 0.89 | |
| | 0.88 | |
| | 1.07 | |
| HOE | 0.30 | 0.27 ± 0.02 |
| | 0.24 | |
| | 0.23 | |
| | 0.34 | |
| | 0.24 | |

When the extracellular pH changed from 6.4 to 7.4, there was a reduction by a factor of 3.6 in the rate of increase in intracellular pH compared with the control. Thus, it is possible by using cariporide during reperfusion to reduce significantly the rate of realkalinization.

### EXAMPLE 2

### Measurement of vWF Release After Reperfusion

The measurements were carried out in a Heraeus Heracell incubator. This made it possible to calculate the umbilical vein endothelial cells under controlled physiological conditions (temperature 37° C., relative humidity 100%, pCO₂ constant at 5%), and to ensure rapid change of different cell culture media.

Said cells were initially incubated with acidotic medium (pH 6.4 composed of the ingredients: medium M199 w/Earle's & amino acids, w/L-glutamine, w/o NaHCO₃, w/o Hepes+0.084 g NaHCO₃/1) or pH standard medium (pH 7.4 composed of the ingredients: medium M199 w/Earle's & amino acids, w/L-glutamine, w/o NaHCO₃, w/o Hepes+2.200 g NaHCO₃/1), for one, three or 48 hours. Before starting the reperfusion, samples of supernatant were taken to determine the vWF concentration under acidotic conditions (vWF_{acidosis}) and control conditions (vWF_{co}). To simulate reperfusion, the medium was changed to one with a pH of 7.4 (ingredients: medium M199 w/Earle's & amino acids, w/L-glutamine, w/o NaHCO₃, w/o Hepes+2.200 g NaHCO₃/1+10 μM histamine) to which the NHE inhibitor cariporide was added in a concentration of 10 μM. Change to the same medium without corresponding addition of inhibitor served as control.

The samples taken from the supernatant were used to determine the vWF concentration. This was done by an ELISA method (enzyme-linked immuno sorbent assay) using specific antibodies. The vWF content of standard human plasma (Behring, Marburg) is calculated using an international standard (2^{nd} International Standard 87/718, National Institute for Biological Standards and Control, London).

Table 3: vWF concentration in the cell supernatant under acidotic (vWF_{acidosis}) and under control conditions (vWF_{co}), measured after incubation for 15 minutes. The vWF concentration under control conditions is set at 100%.

TABLE 3

| | |
|---|---|
| vWF_{co} | 100% |
| vWF_{acdosis} (constitutive) | 46 ± 1.1% |
| vWF_{acidosis} (stimulated, histamine 50 μM) | 52 ± 2.5% |

The acidosis led to a distinct decrease in vWF secretion, both the constitutive secretion and the stimulated Weibel-Palade body secretion. The vWF secretion was reduced by a factor of 2 compared with control cells during acidosis (pHₑₓ=6.4).

Table 4: vWF secretion was measured during a 10-minute reperfusion time with stimulation. The vWF secretion of the control cells (vWF_{co}) was set at 100%. The vWF concentration during the reperfusion of preacidotic cells (vWF_{acidosis}) and the vWF concentration during reperfusion of preacidotic cells in the presence of 10 μM of cariporide (vWF_{HOE}) have been indicated as values relative to the control values. Control cells were incubated with cariporide (vWF_{co+HOE})

TABLE 4

| | |
|---|---|
| vWF_{co} | 100% |
| vWF_{co+HOE} | 106 ± 3.0% |
| vWF_{acidosis} | 193 ± 8.0% |
| vWF_{HOE} | 139 ± 16% |

During the reperfusion, there was a large increase in vWF secretion by a factor of 2. Blockade of the NHE with cariporide reduces the increased vWF secretion by almost 60% and thus approaches the control values. Control cells incubated with cariporide (10 μM) showed no increase or decrease in vWF secretion.

The foregong examples show that extracellular acidosis, as present, for example, during ischemia leads to an intracellular acidosis, resulting in reduced (constitutive and stimulated) vWF secretion and a reduced P-selectin expression. The subsequent reperfusion and stimulation of the endothelial cells brought about rapid intracellular realkalinization. There was a simultaneous great enhancement of the increased vWF secretion and increased P-selectin expression. A delay of the realkalinization with cariporide reduced the increased vWF secretion and P-selectin expression and, thus, the possible thrombosis and inflammatory reactions. The examples showed that the intracellular pH is determined by the extracellular pH. Secretion by the endothelial cells is, in turn, determined by the intracellular pH. It is thus possible, by inhibiting realkalinization, to reduce greatly the known endothelial cell activation during the reperfusion phase and the worry, connected therewith, about rethrombosis (vWF secretion) and inflammation. Incubation of healthy, non-acidotic control cells with cariporide showed no effect. This indicates a low potential for side effects and prevents an excessive tendency to bleeding. The agent acts only where ischemia is present.

1. A method for the prophylaxis and therapy of acute or chronic diseases which are caused by elevated levels of von Willebrand factors in the blood and/or increased expression of P-selectin, this method comprising administering to a patient in need thereof an effective amount of an inhibitor of the sodium/hydrogen exchanger.

2. The method as claimed in claim 1, wherein at least one of the following compounds is employed as said inhibitor of the sodium/hydrogen exchanger: 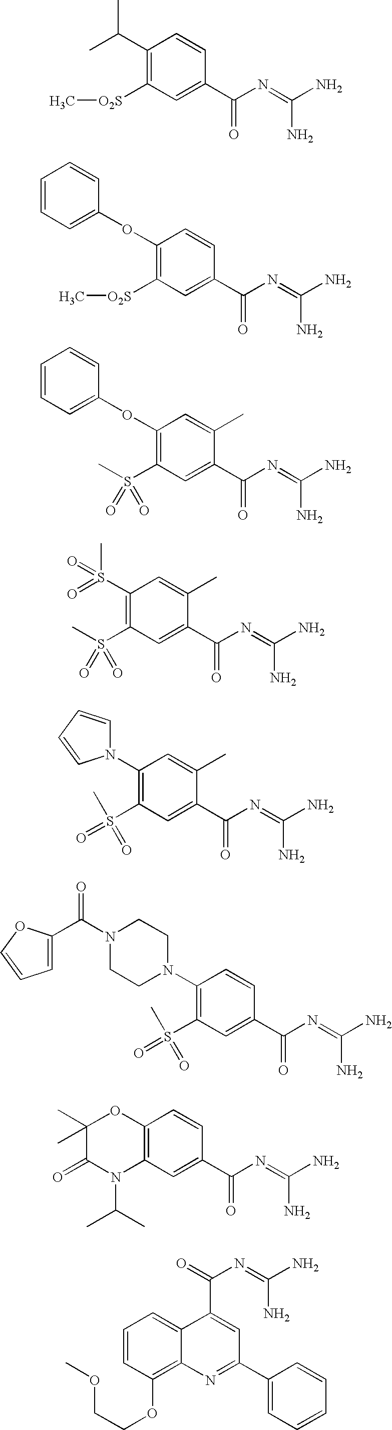 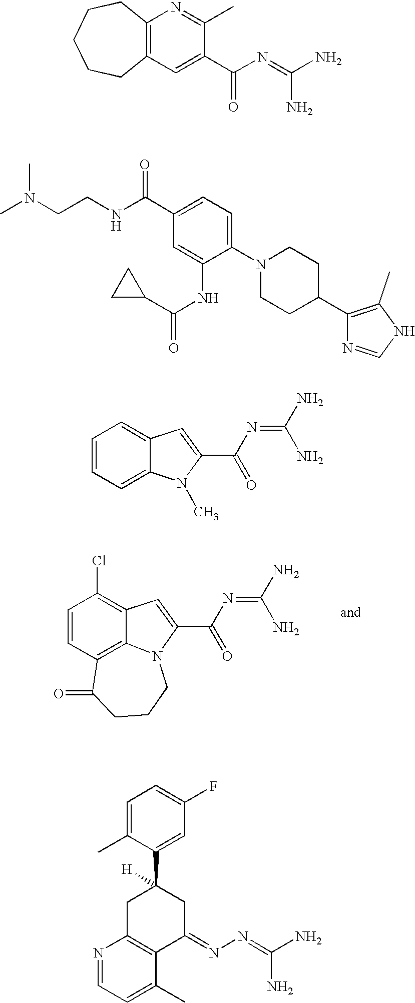
and/or a stereoisomeric form of the abovementioned compounds and/or mixtures of these forms in any ratio, and/or of the physiologically tolerated salts of the abovementioned compounds.

3. The method as claimed in claim 1, wherein cariporide 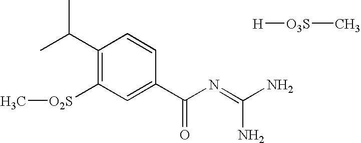
is employed as said inhibitor of the sodium/hydrogen exchanger.

4. The method as claimed in claim 1, wherein the disorder is selected from a thrombotic disorder which is provoked by ischemic states with subsequent reperfusion; a thrombotic disorder occurring during or after surgical operations; pulmonary embolisms; a deep vein thrombosis and inflammatory disorders as might occur during ischemia and subsequent reperfusion, during vasculitis such as associated with an autoimmune disease or connective tissue disease, or an incipient inflammatory reaction, prophylaxis and treatment of arteriosclerosis, prophylaxis and treatment of cancer or treatment of inflammations of joints and arthritic disorders such as rheumatoid arthritis.

5. The method as claimed in claim 1, wherein said inhibitor of the sodium/hydrogen exchanger is employed in combination with at least one anticoagulant, a platelet aggregation-inhibiting or fibrinolytic agent.

6. The method as claimed in claim 5, wherein the additional agent is selected from the group consisting of factor Xa inhibitors, standard heparin, a low molecular weight heparin, a direct thrombin inhibitor, a fibrinogen receptor antagonist, streptokinase, urokinase and tissue plasminogen activator.

7. The method as claimed in claim 6, wherein said low molecular weight heparin is selected from enoxaparin, dalteparin, certroparin, parnaparin and tinzaparin.

8. The method as claimed in claim 6, wherein said thrombin inhibitor is hirudin or aspirin.

9. The method as claimed in claim 1, wherein the agents are administered by oral, inhalational, rectal or transdermal administration or by subcutaneous, intraarticular, intraperitoneal or intravenous injection.
